(19) United States
(12) Patent Application Publication
(10) Pub. No.: US 2018/0208528 A1
(43) Pub. Date: Jul. 26, 2018

(54) PROCESS FOR THE CO-PRODUCTION OF 2,3,3,3-TETRAFLUOROPROPENE AND 1,3,3,3-TETRAFLUOROPROPENE

(71) Applicant: MEXICHEM FLUOR S.A. DE C.V., SAN LUIS POTOSI (MX)

(72) Inventors: CLIVE ROBERT GIDDIS, CHESHIRE (GB); FIONA LOUISE SMITH, CHESHIRE (GB); JONATHAN JUNHAY MAN, CHESHIRE (GB); CARA JOY CRONSHAW, CHESHIRE (GB)

(73) Assignees: MEXICHEM FLUOR S.A. DE C.V., SAN LUIS POTOSI (MX); MEXICHEM UK LIMITED, RUNCORN CHESHIRE (GB)

(21) Appl. No.: 15/745,471

(22) PCT Filed: Jul. 14, 2016
(86) PCT No.: PCT/GB2016/052146

§ 371 Date: Jan. 17, 2018

(30) Foreign Application Priority Data
Jul. 17, 2015 (GB) 1512591.7

(51) Int. Cl. C07C 17/358; C07C 17/25; C07C 17/21; C07C 19/10; C07C 21/18

(52) U.S. Cl. CPC C07C 17/358; C07C 17/25; C07C 17/21; C07C 2523/26; C07C 21/18; C07C 2523/06; C07C 19/10

(57) ABSTRACT

The present invention provides a method of producing 2,3,3,3-tetrafluoropropene (HFO-1234yf), wherein the method comprises two or more reaction steps, at least one of said reaction steps comprising the production of 1,3,3,3-tetrafluoropropene (HFO-1234ze) and/or one or more HFO-1234ze precursors from one or more HFO-1234yf precursors, wherein at least a portion of the HFO-1234ze is recovered.

The present invention concerns a process for the production of 2,3,3,3-tetrafluoropropene (HFO-1234yf) and in particular a method for the coproduction of 1,3,3,3-tetrafluoropropene (HFO-1234ze) and HFO-1234yf.

2,3,3,3-tetrafluoropropene is also known as HFO-1234yf, HFC-1234yf or simply 1234yf. Hereinafter, unless otherwise stated, 2,3,3,3-tetrafluoropropene will be referred to as HFO-1234yf. The known processes for preparing 1234yf typically suffer from disadvantages such as low yields, and/or the handling of toxic and/or expensive reagents, and/or the use of extreme conditions, and/or the production of toxic by-products. Methods for the preparation of 1234yf have been described in, for example, Journal Fluorine Chemistry (82), 1997, 171-174. In this paper, 1234yf is prepared by the reaction of sulphur tetrafluoride with trifluoroacetylacetone. However, this method is only of academic interest because of the hazards involved in handling the reagents and their expense. Another method for the preparation of 1234yf is described in U.S. Pat. No. 2,931,840. In this case, pyrolysis of C1 chlorofluorocarbons with or without tetrafluoroethylene was purported to yield 1234yf. However, the yields described were very low and again it was necessary to handle hazardous chemicals under extreme conditions. It would also be expected that such a process would produce a variety of very toxic by-products.

1,3,3,3-tetrafluoropropene (or HFO-1234ze), as is HFO-1234yf, is useful as a refrigerant or heat transfer agent as well as a blowing agent or propellant, particularly as a replacement for hydrofluorocarbon (HFC) compounds which despite their low ozone depletion potential have a high global warming potential. HFO-1234yf and HFO-1234ze each have the advantage of low ozone depletion potential and low global warming potential, making them particularly attractive in such applications.

Accordingly, there is a need for a process which is able to produce both HFO-1234yf and HFO-1234ze, which involves the use of widely available feedstocks, produces a commercially acceptable yield and does not produce large quantities of hazardous chemicals which require disposal. The present invention addresses this problem.

The listing or discussion of a prior-published document in this specification should not necessarily be taken as an acknowledgement that the document is part of the state of the art or is common general knowledge.

In a first aspect, the invention provides a method of producing 2,3,3,3-tetrafluoropropene (HFO-1234yf), wherein the method comprises two or more reaction steps, at least one of said reaction steps comprising the production of 1,3,3,3-tetrafluoropropene (HFO-1234ze) and/or one or more HFO-1234ze precursors from one or more HFO-1234yf precursors, wherein at least a portion of the HFO-1234ze is recovered and at least a portion of the HFO-1234yf is recovered;

wherein the HFO-1234yf precursors comprise compounds according to the formula

F₃C—CXX—CH₂X

where each X is independently H or a halogen and wherein at least 2 X groups are a halogen; or

F₃C—CX═CH₂

where X is a halogen; and
wherein the HFO-1234ze precursors comprise compounds according to the formula

F₃C—CH₂—CHX₂

where each X is independently a halogen; or

F₃C—CH═CHX

where X is a halogen.

The inventors have surprisingly found that one or more of the steps required to efficiently produce HFO-1234yf also provide HFO-1234ze and/or one or more of its precursors, generally as a minor proportion of the relevant product stream. Even when the quantity of these products that are generated is small, by careful treatment of the various product streams and arrangement of the process in accordance with the invention, the inventors have found that it is possible to manufacture significant quantities of HFO-1234ze simultaneously with the production of HFO-1234yf. This presents significant advantages to the manufacturer in terms of both the provision for efficient use of reaction by-products and ability to produce more than one product from a single manufacturing plant.

Preferably the halogens are chlorine or fluorine.

Preferably, wherein the HFO-1234ze is recovered at the end of the process as a minor product, for example wherein the ratio of production by weight of HFO-1234yf to HFO-1234ze is 99.5-80:0.5-20, for example 99.5-90:0.5-10, e.g. 95-99:1-5.

In preferred embodiments, the HFO-1234yf precursors comprise one or more compounds selected from 1,1,1-trifluoro-2,3-dichloropropane (HCFC-243db), 1,1,1,2,2-pentafluoropropane (HFC-245cb), 2-chloro-1,1,1,2-tetrafluoropropane (HCFC-244bb), 3-chloro-1,1,1,2-tetrafluoropropane (HCFC-244eb), 1,1,1,2,3-pentafluoropropane (HFC-245eb), 2-chloro-3,3,3-trifluoropropene (HCFO-1233xf).

In preferred embodiments, the HFO-1234ze precursors comprise one or more compounds selected from 1,1,1,3,3-pentafluoropropane (HFC-245fa), 3-chloro-1,1,1,3-tetrafluoropropene (HCFC-244fa), 3,3-dichloro-1,1,1-trifluoropropane (HCFC-243fa), 1-chloro-3,3,3-trifluoropropene (HCFO-1233zd).

It is preferred that the HFO-1234ze recovered at the end of the process has an E:Z isomer ratio of at least 95:5, for example at least 99:1 or most preferably consists of HFO-1234zeE. Where HFO-1234zeE is recovered preferentially (e.g. where the ratio of E:Z isomers recovered is greater than 95:5), it is also preferred that the HFO-1234ze precursors include HFO-1234zeZ.

In some embodiments, at least a portion of the one or more of the HFO-1234ze precursors following one or more of the reaction steps is passed directly or indirectly to the or a following reaction step, if present, and/or is recycled to a preceding reaction step, if present.

In preferred embodiments, one of the reaction steps “A” comprises exposing a feed stream “A” comprising HFO-245cb to conditions suitable for dehydrofluorination to produce a product stream “A” which comprises HFO-1234yf. Preferably, the product stream “A” comprises HFO-1234ze (e.g. HFO-1234zeE) and/or one or more HFO-1234ze precursors and/or the feed stream “A” comprises HFO-1234ze, e.g. HFO-1234zeE as a minor proportion but preferably substantially no HFO-1234ze precursors.

Preferably, the product stream “A” is passed directly or indirectly to a separation train to recover HFO-1234yf and, if present, HFO-1234ze (e.g. HFO-1234zeE) and, if present, one or more of the HFO-1234ze precursors. It is preferred that at least a portion of the HFO-1234ze precursors recycled from product stream “A” are recycled to one or more earlier reaction steps. Preferably, HFO-1234yf precursors are also recycled to one or more earlier reaction steps.

In some embodiments, the conditions in reaction step “A” suitable for dehydrofluorination of HFC-245cb comprise exposure to a catalyst “A”, preferably in the vapour phase, preferably at a temperature between 0° C. and 500° C., for example between 250° C. and 400° C., preferably at a pressure between 0.1 barg and 30 barg, for example between about 1 barg and about 10 barg, e.g. between about 1 barg and about 4 barg.

The catalyst used in the reaction step “A” may be any suitable catalyst that is effective to dehydrofluorinate HFC-245cb to HFO-1234yf. Preferred catalysts are bulk form or supported catalysts comprising activated carbon, a zero-valent metal, a metal oxide, a metal oxyhalide, a metal halide, or mixtures of the foregoing as described above in relation to the catalyst for the catalytic dehydrohalogenation step

Preferred catalysts for catalytic dehydrofluorination HFC-245cb to HFO-1234yf are those which comprise chromia, alone or chromia that has been modified by the addition of Zn, Mn, Zr, In, Ni, Al and/or Mg and/or a compound of one or more of these metals. A preferred chromia-based catalyst for use in the reaction step “A” is a zinc/chromia catalyst. The same catalyst (e.g. a chromia-based catalyst) may be used for the catalytic dehydrochlorination and hydrofluorination steps.

Preferably, the HFO-1234ze precursors in product stream “A” comprise HFC-245fa and/or HFO-1234zeZ.

In a further embodiment, the method comprises a reaction step “B” which comprises contacting a feed stream “B” comprising HCFO-1233xf with HF in conditions sufficient to hydrofluorinate HCFO-1233xf to form a product stream “B” comprising HFC-245cb, e.g. for provision directly or indirectly to the feed stream “A”.

It is preferred that the product stream “B” comprises HFO-1234ze and/or one or more HFO-1234ze precursors. In some embodiments, the product stream “B” comprises HFO-1234yf, for example as a major or a minor component. The feed stream “B” may comprise HFO-1234ze and/or one or more of the HFO-1234ze precursors. In preferred embodiments, at least a portion of the HFO-1234ze precursors in the feed stream “B” are recycled from one or more of products stream “A” and/or product stream “B”, for example through the separation train.

In preferred embodiments, the product stream “B” is passed directly or indirectly to a separation train to recover HFO-1234yf and, if present, HFO-1234ze (e.g. HFO-1234zeE) and, if present, one or more of the HFO-1234ze precursors. Preferably, the product stream “B” is passed directly or indirectly to a separation train to separate HFC-245cb, e.g. for provision directly or indirectly to the feed stream “A”.

In preferred embodiments, the conditions of reaction step B are sufficient to convert at least a portion of one or more of the HFO-1234ze precursors to HFO-1234ze (e.g. by dehydrohalogenation such as dehydrofluorination and/or dehydrochlorination) and/or to convert a at least a portion of one or more of the HFO-1234ze precursors to a different HFO-1234ze precursor (e.g. by hydrofluorination).

Preferably, the HFO-1234ze precursors in product stream “B” comprise one or more of HFC-245fa, HCFC-244fa, HCFC-243fa, HCFO-1233zd and/or HFO-1234zeZ. Preferably, the HFO-1234ze precursors in feed stream “B” comprise one or more of HFC-245fa, HCFC-244fa, HCFO-1233zd and/or HFO-1234zeZ.

In preferred embodiments, the conditions in reaction step “B” comprise contacting the feed stream “B” with HF, preferably in the presence of a catalyst “B”, preferably at a temperature between about 0 to about 450° C. and a pressure of from about 0.1 to about 30 bara, more preferably at a temperature of from about 200 to about 400° C. and more preferably at a pressure of from 1 to about 20 bara, most preferably at a temperature of from about 300 to about 380° C. and a pressure of from 5 to about 20 bara. The catalyst “B” may comprise a catalyst such as those described in relation to step “A”. It is preferred that catalyst “B” comprises a zinc/chromia catalyst.

In preferred embodiments, the method comprises a reaction step “C” which comprises exposing a feed stream “C” comprising HCFC-243db to conditions suitable for dehydrochlorination to form a product stream “C” comprising HCFO-1233xf, e.g. for provision directly or indirectly to the feed stream “B”.

Preferably, the product stream “C” comprises HFO-1234ze and/or one or more HFO-1234ze precursors. Preferably, the product stream “C” comprises HFO-1234yf, preferably as a minor component.

In preferred embodiments, the product stream “C” is passed directly or indirectly to a separation train to recover HFO-1234yf and, if present, HFO-1234ze (e.g. HFO-1234zeE) and, if present, one or more of the HFO-1234ze precursors.

Preferably, the feed stream “C” comprises HFO-1234ze and/or one or more of the HFO-1234ze precursors. In some embodiments, at least a portion of the HFO-1234ze precursors in the feed stream “C” are recycled from one or more of products stream “A” and/or product stream “B” and/or product stream “C”, for example through the separation train.

In preferred embodiments, the conditions of reaction step “C” are sufficient to convert at least a portion of one or more of the HFO-1234ze precursors to HFO-1234ze (e.g. by dehydrohalogenation such as dehydrofluorination and/or dehydrochlorination) and/or to convert at least a portion of one or more of the HFO-1234ze precursors to a different HFO-1234ze precursor (e.g. by hydrofluorination).

Preferably, the HFO-1234ze precursors in product stream “C” comprise one or more of HFC-245fa, HCFC-244fa, HCFC-243fa, HCFO-1233zd and/or HFO-1234zeZ. Preferably, the HFO-1234ze precursors in feed stream “C” comprise one or more of HFC-245fa, HCFC-244fa, HCFO-1233zd and/or HFO-1234zeZ.

In preferred embodiments, the conditions in reaction step “C” comprise contacting the feed stream “C” in the presence of a catalyst “C”, preferably at a temperature between about 0 to about 450° C. and a preferably at pressure of from about 0.1 to about 30 bara, more preferably at a temperature of from about 200 to about 400° C. and more preferably at a pressure of from 1 to about 20 bara, most preferably at a temperature of from about 300 to about 380° C. and a pressure of from 5 to about 20 bara. The catalyst “C” may comprise a catalyst such as those described in relation to step “A”. It is preferred that catalyst “C” comprises a zinc/chromia catalyst.

In some embodiments, steps “B” and “C” are conducted simultaneously in the same reactor.

In preferred embodiments, the method comprises a reaction step “D” which comprises exposing a feed stream “D” comprising 3,3,3-trifluoropropene (HFO-1243zf) to a stream of Cl₂ in conditions suitable for fluorination to form a product stream “D” comprising HCFC-243db, e.g. for provision directly or indirectly to the feed stream “C”.

Preferably, the product stream “D” comprises one or more HFO-1234ze precursors. Preferably, the HFO-1234ze precursors in product stream “D” comprise HCFC-243fa.

It is preferred that the reaction step “D” comprises exposing the feed stream “D” to the Cl₂ in the presence of a catalyst “D”, the catalyst preferably comprising one or more transition metals and the Cl₂ preferably being present in a molar excess in relation to the 1243zf. In most preferred embodiments, the catalyst comprises one or more elemental transition metals and or compounds of transition metals (e.g. chlorides and/or oxides), preferably supported on one or more metal oxides, for example alumina.

The reaction step “D” is preferably performed at a temperature between about 100° C. and about 400° C. and preferably at a pressure between about 0 barg and about 30 barg, more preferably at a temperature of 150° C. to 300° C. and a pressure of about 5 barg to 10 barg.

In a further aspect, the invention provides a method of producing 2,3,3,3-tetrafluoropropene (HFO-1234yf) comprising contacting a feed stream comprising 1,1,1,2,2-pentafluoropropane (HFC-245cb) with a catalyst under conditions suitable to dehydrofluorinate HFC-245cb to form a product stream comprising HFO-1234yf in a major proportion and HFO-1234ze (e.g. HFO1234zeE) in a minor proportion.

Preferably, the product stream further comprises one or more HFO-1234ze precursors according to the formula

F₃C—CH₂—CHX₂

where each X is a halogen; or

F₃C—CH═CHX

where X is a halogen.

The inventors have surprisingly found that the dehydrofluorination of HFC-245cb can produce significant quantities of HFO-1234ze and its precursors in addition to the expected HFO-1234yf.

Preferably the halogens are chlorine or fluorine.

In preferred embodiments, the HFO-1234ze precursors comprise one or more compounds selected from 1,1,1,3,3-pentafluoropropane (HFC-245fa), 3-chloro-1,1,1,3-tetrafluoropropene (HCFC-244fa), 3,3-dichloro-1,1,1-trifluoropropane (HCFC-243fa), 1-chloro-3,3,3-trifluoropropene (HCFO-1233zd).

In preferred embodiments, the HFO-1234ze precursors include HFO-1234zeZ.

The catalyst preferably comprises any suitable catalyst that is effective to dehydrofluorinate HFC-245cb to HFO-1234yf. Preferred catalysts are bulk form or supported catalysts comprising activated carbon, a zero-valent metal, a metal oxide, a metal oxyhalide, a metal halide, or mixtures of the foregoing as described above in relation to the catalyst for the catalytic dehydrochlorination step.

Preferred catalysts for catalytic dehydrofluorination HFC-245cb to HFO-1234yf are those which comprise chromia, alone or chromia that has been modified by the addition of Zn, Mn, Zr, In, Ni, Al and/or Mg and/or a compound of one or more of these metals. A preferred chromia-based catalyst for use in the reaction is a zinc/chromia catalyst. The same catalyst (e.g. a chromia-based catalyst) may be used for the catalytic dehydrochlorination and hydrofluorination steps.

It is preferred that the feed stream contacted with the catalyst in the vapour phase, preferably at a temperature between 0° C. and 500° C., for example between 250° C. and 400° C., preferably at a pressure between 0.1 barg and 30 barg, for example between about 1 barg and about 10 barg, e.g. between about 1 barg and about 4 barg.

In some embodiments, the feed stream comprises HFO-1234ze (e.g. HFO-1234zeE), for example in a proportion of less than 5 wt % of the feed stream, for example less than about 2 wt % or less than about 1 wt % of the feed stream. Preferably, the product stream comprises a greater proportion of HFO-1234ze (e.g. HFO-1234zeE) than does the feed stream.

Preferably, the product stream comprises at least 0.1 wt % HFO-1234ze (e.g. HFO-1234zeE), for example at least 0.5 wt % HFO-1234ze (e.g. HFO-1234zeE) or at least 1 wt % HFO-1234ze (e.g. HFO-1234zeE).

Preferably, the product stream comprises at least about 40 wt % HFO-1234yf, for example at least about 50 wt % HFO-1234yf or at least about 60 wt % HFO-1234yf.

Embodiments of the present invention will be described with reference to the following drawings and examples:

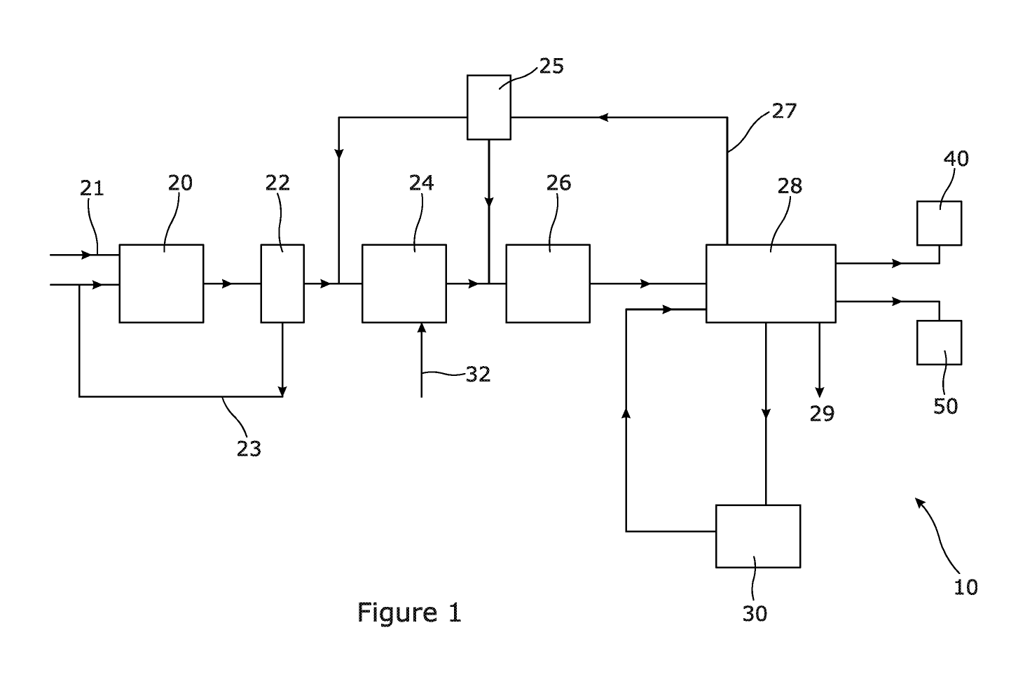

FIG. 1 shows a schematic diagram of an integrated process according to the present invention.

An integrated process 10 is shown schematically in FIG. 1. The process is for the coproduction of HFO-1234yf and HFO-1234zeE. The process utilises a sequence of reactor D 20, reactor C 24, reactor B 26 and reactor A 30, with first and second separation trains 22, 28 being positioned between the reactor D 20 and reactor C 24 and the reactor B 26 and reactor A 30 respectively. A Cl₂ supply 21 is provided into the reactor D 21 and a HF supply line 32 is provided into the reactor C 24. The first separation train 22 has a first recycle line 23 for recycling components to the reactor D 20 and the second separation train 28 has a waste outlet 29, a second recycle line 27 for recycling components to the reactor C 24 and reactor B 26 via a distillation column 25 and lines to a HFO-1234yf collection tank 40 and a HFO-1234zeE collection tank 50. The process is arranged to produce HFO-1234yf as a major component of its output, the production of HFO-1234zeE being a minor component.

In use, a supply of HFO-1243zf is charged to the reactor D 20, along with a supply of Cl₂ 21, in a molar ratio of at least about 1:2 HFO-1234zf:Cl₂. The reactor D 20 preferably contains a transition metal containing catalyst, such as 10 wt % Cu on Al₂O₃ and is preferably heated to around 200° C. at 8 barg. The reaction produces a product stream which comprises HCFC-243db as a major component and one or more 1234ze precursors, such as HCFC-243fa and HCFC-244fa as minor components. Some other HFO-1234yf precursors, such as HCFO-1233xf may also be produced.

The product stream is passed from the reactor D 20 into the first separation train 22 to separate the product stream from unreacted Cl₂, trace HCl and HFO-1243zf, which is recycled to the reactor D 20 through the first recycle line 23. The product stream is then passed as a feed stream into the reactor C 24.

The reactor C 24 is primarily adapted to dehydrochlorinate the HCFC-243db in its feed stream to form a product stream which comprises HCFO-1233xf. However, in addition to the HFO-1234ze precursors formed in the reaction vessel D 20, the feed stream of the reaction vessel C 24, also contains previously unreacted HCFC-243db, which is recycled through the second recycle line, having been separated from the HFO-1234ze precursors in the second recycle line 27 in the distillation column 25. The reactor C may contain a zinc/chromia catalyst and may be operated at a temperature of around 350° C. and a pressure of about 15 barg The HF supply 32 is provided into reactor C 24 to reduce the fouling of the catalyst and also to supply HF to the following hydrofluorination reaction in reactor B 26.

The product stream comprises HCFO-1233xf as a major component, however the product stream also includes HFO-1234zeE as a minor component and additionally contains one or more HFO-1234ze precursors such as HCFC-243fa, HCFC-244fa, HCFO-1233zd, HFC-245fa and HFO-1234zeZ, some of which are formed in reactor C 24. It is understood that the HFO-1234zeE and precursors thereof are produced both as by-products of the HFC-243db in the product feed, but also from reactions of the HFO-1234ze precursors in the product feed. HFO-1234yf may also be formed in reactor C 24.

The product stream of the reactor C 24 is provided as a feed stream to the reactor B 26, which is primarily adapted to hydrofluorinate the HCFO-1233xf in the feed stream to form a product stream comprising HFC-245cb. A portion of the feed stream is also provided via the second recycle line 27 from the separation train 28, that portion generally comprising HFO-1234ze precursors and HFO-1234yf precursors other than HCFC243db, following separation in the distillation column 25. The feed stream is contains some or all of the HF provided to the reactor C 24 by the HF supply 32. It is preferably contacted with a catalyst such as a zinc/chromia catalyst. The reaction is preferably performed at a temperature of around 350° C. and a pressure of about 15 barg. The product stream comprises HFC-245cb as a major component, however the product stream also includes HFO-1234zeE as a minor component and additionally contains one or more HFC-1234ze precursors such as HCFC-243fa, HCFC-244fa, HCFO-1233zd, HFC-245fa and HFO-1234zeZ, some of which are formed in reactor B 26. It is understood that the HFO-1234zeE and precursors thereof are produced both as by-products of the HCFO-1233xf in the product feed, but also from reactions of the HFO-1234ze precursors in the product feed. HFO-1234yf is also formed in reactor B.

The product stream of the reactor B 26 is then fed into the separation train 28 for separation and cleaning of the various products. The separation train 28 removes light organics and bulk HF and/or HCl from the product stream and also removes HFO-1234ze precursors and HFO-1234yf precursors from the product stream and passes them through the recycle line 27 to the reactor C 24 or reactor B 26. The remaining materials are scrubbed to remove HF and/or HCl and passed for further separation. HFO-1234yf and HFO-1234zeE are recovered separately for storage, while HFC-245cb (which may contain some impurities, for example, HFO-1234zeE) is passed as a feed stream to the reactor A 30. Any materials which cannot be recycled are passed to waste via the waste stream 29.

The reactor A 30 is primarily arrange to dehydrofluorinate the HFC-245cb to form HFO-1234yf. The feed stream containing HFC-245cb is preferably contacted in the reactor A 30 with a zinc chromia catalyst at a preferred temperature of about 350° C. and a preferred pressure of about 2 barg. The product stream contains HFO-1234yf as a major component and also contains HFO-1234zeE as a minor component, together with one or more HFO-1234ze precursors such as HFC-245fa and HFO-1234zeZ. The product stream is passed to the separation train 28 and separated simultaneously and in the same manner as the product stream of the reactor B 26.

The overall process 10 produces both HFO-1234yf and HFO-1234zeE in commercial quantities, the ratio of production of HFO-1234yf:HFO-1234zeE preferably being in the order of 99.5-95:0.5-5 by weight.

### EXAMPLE 1

A 100 ml Inconel reaction tube was charged with 16 g zinc chromia catalyst, fluorinated and then provided with a feed of substantially pure HFC-245cb (approximately 99.65 wt %). The reactions were performed at varying temperatures and at a pressure of 2 barg. The reactor out gas (ROG) was measured and the results are presented in Table 1 below.

TABLE 1

| | 245cb | Av. flow | ROG/wt % | | ROG/ppm | | | | |
|---|---|---|---|---|---|---|---|---|---|
| Temp/° C. | conversion/% | ml/min | 1234yf | 245cb | 1234zeE | 245fa | 1234zeZ | 143a | 1243zf |
| 250 | 12.07 | 27.40 | 12.00 | 87.93 | 15 | 31 | 7 | 8 | 229 |
| 275 | 17.73 | 29.38 | 17.64 | 82.27 | 93 | 116 | 16 | 30 | 191 |
| 300 | 26.15 | 29.50 | 25.97 | 73.85 | 479 | 414 | 109 | 77 | 173 |
| 325 | 50.13 | 27.35 | 49.12 | 49.87 | 4970 | 3083 | 1177 | 205 | 235 |
| 350 | 62.75 | 29.18 | 59.76 | 37.25 | 17792 | 6349 | 4484 | 246 | 263 |

The above results surprisingly show that a significant quantity of HFO-1234zeE (e.g. around 1 wt % to 2 wt % ROG) can be produced by exposing HFC-245cb to dehydrofluorination conditions. Moreover, a significant quantity (e.g. around 0.5 wt % to 1.5 wt % ROG) precursors of HFO-1234zeE, for example HFO-1234zeZ and HFC-245fa, may be produced under the same conditions.

### EXAMPLE 2

A series of two reactors (each 25 ml Inconel reactor) is connected and each is charge with 6 g/5.5 ml of a zinc/chromia catalyst of particle size 2.0-3.65 mm and fluorinated. The reactor C is for the conversion of HCFC-243db to HCFO-1233xf, the reactor B is for the conversion of HCFO-1233xf to HFC-245cb.

Each reactor is set to 350° C. and 15 barg. The feeds and reactor out gas compositions are shown in Tables 2 to 5.

### Reactor Feed Composition

TABLE 2
Reactor C-

| Feed (in) | Mole Ratio |
|---|---|
| HF | 9 |
| 243db | 1 |

TABLE 3
Reactor B-

| Feed (in) | Mole Ratio |
|---|---|
| Air | 0.4 |
| HF | 9 |
| 1233xf | 1 |

### Reactor Outlet Composition

TABLE 4
Reactor C

| Feed (out) | Mole Ratio |
|---|---|
| Air | 0.4 |
| HF | 9 |
| 243db | 0.02 |
| 1233xf | 0.88 |
| 1234yf | 0.05 |
| HCl | 0.98 |
| 143a, 1234zeE, 236fa, | 0.05 |
| 1234ZeZ, 244bb, 245fa, | |
| 245ca, 1233zdZ, 1233zdE, | |
| other 244 isomers, other 243 | |
| isomers | |

TABLE 5
Reactor B

| Feed (out) | Mole Ratio |
|---|---|
| Air | 0.4 |
| HF | 8.6 |
| 243db | 0.02 |
| 1233xf | 0.58 |
| 245cb | 0.30 |
| 1234yf | 0.08 |
| HCl | 0.20 |
| 143a, 1234zeE, 236fa, 1234ZeZ, 244bb, | 0.04 |
| 245fa, 245ca, 1233zdZ, 1233zdE, other | |
| 244 isomers, other 243 isomers | |

As the results in Tables 4 and 5 show, significant impurities, including HFO-1234zeE and its precursor compounds, are produced in these reactions.

### EXAMPLE 3

A 25 ml glass lined stainless steel reaction tube was charged with 7 ml or 4 g 10% Cu/Al₂O₃ catalyst, and fed with a stream of HFO-1243zf and Cl₂ in a molar ratio of 4:1 at atmospheric pressure. The reaction temperatures and the organic compounds found in the product stream are set out in Table 6.

TABLE 6

| | Reactor Temp ° C. | | | | | | |
|---|---|---|---|---|---|---|---|
| | 75 | 100 | 125 | 150 | 175 | 200 | 225 |
| Compound | Mole % | Mole % | Mole % | Mole % | Mole % | Mole % | Mole % |
| 1243zf | 0.24 | 0.15 | 0.12 | 0.00 | 0.00 | 0.00 | 0.00 |
| 1233xf | 0.24 | 0.29 | 0.82 | 0.86 | 0.41 | 1.14 | 1.26 |
| isomer 244 | 0.00 | 0.00 | 0.00 | 0.00 | 0.00 | 0.60 | 0.92 |
| 244db | 3.89 | 5.68 | 6.74 | 7.33 | 5.42 | 2.47 | 8.22 |
| 243db | 94.82 | 93.54 | 91.33 | 90.34 | 92.46 | 93.20 | 80.70 |
| isomer 1224 | 0.00 | 0.00 | 0.00 | 0.28 | 0.72 | 1.57 | 4.71 |
| 243fa | 0.81 | 0.35 | 0.98 | 1.18 | 0.99 | 0.00 | 0.00 |
| Conversion 1243zf/% | 99.8 | 99.9 | 99.9 | 100.0 | 100.0 | 100.0 | 100.0 |
| Selectivity 243db/% | 95.0 | 93.7 | 91.4 | 90.3 | 92.5 | 93.2 | 80.7 |

The above results demonstrate that a significant quantity of HFO-1234ze precursor compounds are produced in the reaction, in addition to the production of HCFC-243db.

Preferences and options for a given aspect, feature or parameter of the invention should, unless the context indicates otherwise, be regarded as having been disclosed in combination with any and all preferences and options for all other aspects, features and parameters of the invention.

Where a molecule, for example HFO-1234ze, may take the form of E and Z isomers, the general disclosure of that molecule is intended to refer equally to both the E and Z isomers.

The invention is defined by the following claims.

1. A method of producing 2,3,3,3-tetrafluoropropene (HFO-1234yf), wherein the method comprises two or more reaction steps, at least one of said reaction steps comprising the production of 1,3,3,3-tetrafluoropropene (HFO-1234ze) and/or one or more HFO-1234ze precursors from one or more HFO-1234yf precursors, wherein at least a portion of the HFO-1234ze is recovered and at least a portion of the HFO-1234ze precursors following one or more of the reaction steps is passed directly or indirectly to the following or a following reaction step, if present, and/or is recycled to a preceding reaction step if present; and
wherein the HFO-1234yf precursors comprise compounds according to the formula
F₃C—CXX—CH₂X
where each X is independently H or a halogen and wherein at least 2 X groups are a halogen; or
F₃C—CX═CH₂
where X is a halogen; and
wherein the HFO-1234ze precursors comprise compounds according to the formula
F₃C—CH₂—CHX₂
where each X is independently a halogen; or
F₃C—CH═CHX
where X is a halogen,
wherein one of the reaction steps “A” comprises exposing a feed stream “A” comprising HFO-245cb to conditions suitable for dehydrofluorination to produce a product stream “A” which comprises HFO-1234yf and wherein the method further comprises a preceding reaction step “B” which comprises contacting a feed stream “B” comprising HCFO-1233xf with HF in conditions sufficient to hydrofluorinate HCFO-1233xf to form a product stream “B” comprising HFC-245cb.

2. A method according to claim 1, wherein the HFO-1234ze is recovered as a minor product.

3. A method according to claim 1, wherein the ratio of production by weight of HFO1234yf to HFO-1234ze is 99.5-80:0.5-20.

4. A method according to claim 1 wherein the HFO-1234yf precursors comprise one or more compounds selected from 1,1,1-trifluoro-2,3-dichloropropane (HCFC-243db), 1,1,1,2,2-pentafluoropropane (HFC-245cb), 2-chloro-1,1,1,2-tetrafluoropropane (HCFC-244bb), 3-chloro-1,1,1,2-tetrafluoropropane (HCFC-244eb), 1,1,1,2,3-pentafluoropropane (HFC-245eb), 2-chloro-3,3,3-trifluoropropene (HCFO-1233xf).

5. A method according to claim 1, wherein the HFO-1234ze precursors comprise one or more compounds selected from 1,1,1,3,3-pentafluoropropane (HFC-245fa), 3-chloro-1,1,1,3-tetrafluoropropene (HCFC-244fa), 3,3-dichloro-1,1,1-trifluoropropane (HCFC-243fa), 1-chloro-3,3,3-trifluoropropene (HCFO-1233zd).

6. A method according to claim 1 wherein the HFO-1234ze recovered has an E:Z isomer ratio of at least 95:5.

7. A method according to claim 6, wherein the HFO-1234ze precursors include HFO-1234zeZ.

8. A method according to claim 1, wherein the product stream “A” comprises HFO-1234ze and/or one or more HFO-1234ze precursors.

9. A method accordingly to claim 1, wherein the feed stream “A” comprises HFO-1234ze as a minor proportion.

10. A method according to claim 1, wherein the product stream “A” is passed directly or indirectly to a separation train to recover HFO-1234yf and, if present, HFO-1234ze (e.g. HFO-1234zeE) and, if present, one or more of the HFO-1234ze precursors.

11. A method according to claim 10 wherein at least a portion of the HFO precursors recovered from product stream “A” are recycled to one or more earlier reaction steps.

12. A method according to claim 1, wherein the conditions suitable for dehydrofluorination of 245cb comprise exposure to a catalyst “A”.

13. A method according to claim 10, wherein the HFO-1234ze precursors in product stream “A” comprise HFC-245fa and/or HFO-1234zeZ.

14. A method according to claim 1, wherein the product stream “B” comprises HFO-1234ze and/or one or more HFO-1234ze precursors.

15. A method according to claim 1, wherein the product stream “B” comprises HFO-1234yf.

16. A method according to claim 1, wherein the product stream “B” is passed directly or indirectly to a separation train to recover HFO-1234yf and, if present, HFO-1234ze (e.g. HFO-1234zeE) and, if present, one or more of the HFO-1234ze precursors.

17. A method according to claim 1, wherein the product stream “B” is passed directly or indirectly to a separation train to separate HFC-245cb, e.g. for provision directly or indirectly to the feed stream “A”.

18. A method according to claim 1, wherein the feed stream “B” comprises HFO-1234ze and/or one or more of the HFO-1234ze precursors.

19. A method according to claim 18, wherein the conditions of reaction step B are sufficient to convert at least a portion of one or more of the HFO1234ze precursors to HFO-1234ze and/or to convert a at least a portion of one or more of the HFO-1234ze precursors to a different HFO-1234ze precursor.

20. A method according to claim 14, wherein the HFO-1234ze precursors in product stream “B” comprise one or more of HFC-245fa, HCFC-244fa, HCFC-243fa, HCFO-1233zd and/or HFO-1234zeZ.

21. A method according to claim 18, wherein the HFO-1234ze precursors in feed stream “B” comprise one or more of HFC-245fa, HCFC-244fa, HCFO-1233zd and/or HFO-1234zeZ.

22. A method according claim 14, wherein at least a portion of the HFO-1234ze precursors in the feed stream “B” are recycled from one or more of products stream “A” and/or product stream “B”.

23. A method according to claim 1, wherein the conditions in reaction step “B” comprise contacting the feed stream “B” with HF in the presence of a catalyst “B”.

24. A method according to claim 1, comprising a reaction step “C” which comprises exposing a feed stream “C” comprising HCFC-243db to conditions suitable for dehydrochlorination to form a product stream “C” comprising HCFO-1233xf for provision directly or indirectly to the feed stream “B”.

25. A method according to claim 24, wherein the product stream “C” comprises HFO-1234ze and/or one or more HFO-1234ze precursors.

26. A method according to claim 24, wherein the product stream “C” comprises HFO-1234yf.

27. A method according to claim 24, wherein the product stream “C” is passed directly or indirectly to a separation train to recover HFO-1234yf and, if present, HFO-1234ze (e.g. HFO-1234zeE) and, if present, one or more of the HFO-1234ze precursors.

28. A method according to claim 24, wherein the feed stream “C” comprises HFO-1234ze and/or one or more of the HFO-1234ze precursors.

29. A method according to claim 28, wherein at least a portion of the HFO-1234ze precursors in the feed stream “C” are recycled from one or more of products stream “A” and/or product stream “B” and/or product stream “C”.

30. A method according to claim 28, wherein the conditions of reaction step “C” are sufficient to convert at least a portion of one or more of the HFO1234ze precursors to HFO-1234ze and/or to convert a at least a portion of one or more of the HFO-1234ze precursors to a different HFO-1234ze precursor.

31. A method according to claim 25, wherein the HFO-1234ze precursors in product stream “C” comprise one or more of HFC-245fa, HCFC-244fa, HCFC-243fa, HCFO-1233zd and/or HFO-1234zeZ.

32. A method according to claim 28, wherein the HFO-1234ze precursors in feed stream “C” comprise one or more of HFC-245fa, HCFC-244fa, HCFO-1233zd and/or HFO-1234zeZ.

33. A method according to claim 24, wherein the conditions in reaction step “C” comprise contacting the feed stream “C” in the presence of a catalyst “C”.

34. A method according to claim 1, comprising a reaction step “D” which comprises exposing a feed stream “D” comprising 3,3,3-trifluoropropene (HFO-1243zf) to a stream of Cl₂ in conditions suitable for chlorination to form a product stream “D” comprising HCFC-243db.

35. A method according to claim 34, wherein the product stream “D” comprises one or more HFO-1234ze precursors.

36. A method according to claim 35, wherein the HFO-1234ze precursors in product stream “D” comprise HCFC-243fa.

37. A method according to claim 34, wherein the reaction step “D” comprises exposing feed stream “D” to the Cl₂ in the presence of a catalyst “D”, the catalyst preferably comprising one or more transition metals and the Cl₂ preferably being present in a molar excess in relation to the 1243zf.

38. A method of producing 2,3,3,3-tetrafluoropropene (HFO-1234yf) comprising contacting a feed stream comprising 1,1,1,2,2-pentafluoropropane (HFC-245cb) with a catalyst under conditions suitable to dehydrofluorinate HFC-245cb to form a product stream comprising HFO-1234yf in a major proportion and HFO-1234ze in a minor proportion.

39. A method according to claim 38 wherein the product stream further comprises one or more HFO-1234ze precursors according to the formula
F₃C—CH₂—CHX₂
where each X is independently a halogen; or
F₃C—CH═CHX
where X is a halogen.

40. A method according to claim 39, wherein the HFO-1234ze precursors comprise one or more compounds selected from 1,1,1,3,3-pentafluoropropane (HFC-245fa), 3-chloro-1,1,1,3-tetrafluoropropene (HCFC-244fa), 3,3-dichloro-1,1,1-trifluoropropane (HCFC-243fa), 1-chloro-3,3,3-trifluoropropene (HCFO-1233zd).

41. A method according to claim 39, wherein the HFO-1234ze precursors include HFO-1234zeZ.

42. A method according to claim 38, wherein the catalyst comprises a zinc/chromia catalyst.

43. A method according to claim 38, wherein the feed stream is contacted with the catalyst at a temperature between 0° C. and 500° C.

44. A method according to claim 38, wherein the feed stream is contacted with the catalyst at a pressure of from about 0.1 to about 30 bara.

45. A method according to claim 38, wherein the feed stream comprises HFO-1234ze, in a proportion of less than 5 wt % of the feed stream.

46. A method according to claim 45, wherein the product stream comprises a greater proportion of HFO-1234ze than does the feed stream.

47. A method according to claim 38, wherein the product stream comprises at least 0.1 wt % HFO-1234ze.

48. A method according to claim 38, wherein the product stream comprises at least about 40 wt % HFO-1234yf.

49. A method according to claim 12, wherein exposure to Catalyst “A” takes place in the vapour phase.

50. A method according to claim 49 wherein the vapour phase occurs a temperature between 250° C. and 400° C.

51. A method according to claim 49 wherein exposure to catalyst “A” takes place at a pressure between 0.1 barg and 30 barg.

52. A method according to claim 12, where the catalyst “A” comprises a zinc/chromia catalyst.

53. A method according to claim 23 where in contacting the feed stream “B” with HF occurs at a temperature between about 0 to about 450° C. and a pressure of from about 0.1 to 30 bara.

54. A method according to claim 23 where in contacting the feed stream “B” with HF occurs at a temperature between about 300 to 380° C. and a pressure from 5 to about 20 bara.

55. A method according to claim 33, wherein the conditions in reaction step “C” further are at a temperature between about 0 to about 450° C. and a pressure of from about 0.1 to about 30 bara.

56. A method according to claim 33, wherein the conditions in reaction step “C” further are at a temperature of from about 300 to about 380° C. and a pressure of from 5 to about 20 bara.
